(19) United States
(12) Patent Application Publication
(10) Pub. No.: US 2025/0011691 A1
(43) Pub. Date: Jan. 9, 2025

(54) SOLID COMPOSITION

(71) Applicant: GIVAUDAN SA, VERNIER (CH)

(72) Inventors: Emmanuel AUSSANT, Argenteuil (FR); Cindy RADICE PETITPREZ, Argenteuil (FR)

(21) Appl. No.: 18/709,624

(22) PCT Filed: Dec. 5, 2022
(86) PCT No.: PCT/EP2022/084353

§ 371 Date: May 13, 2024

(30) Foreign Application Priority Data
Dec. 6, 2021 (GB) 2117602.9

(51) Int. Cl. C11D 17/00; C11D 3/14; C11D 3/20; C11D 3/22; C11D 3/37; C11D 3/40; C11D 3/50

(52) U.S. Cl. CPC C11D 17/0039; C11D 3/14; C11D 3/2065; C11D 3/222; C11D 3/37; C11D 3/40; C11D 3/505

(57) ABSTRACT

The present invention provides a solid composition consisting of a solid, water-soluble, biodegradable carrier and a microcapsule composition comprising a polymer encapsulating a benefit agent, wherein the benefit agent is encapsulated in core-shell microcapsules comprising a core and a shell surrounding the core. The invention also provides a consumer product comprising the solid composition.

The present invention relates to a solid composition for controlled release of a benefit agent. In particular, the invention is concerned with a solid composition consisting of a solid, water-soluble, biodegradable carrier and a microcapsule composition comprising a polymer encapsulating a benefit agent, wherein the benefit agent is encapsulated in core-shell microcapsules comprising a core and a shell surrounding the core. The invention also relates to a method of making a solid composition as defined herein, to a consumer product comprising the solid composition as defined herein and to the use of the solid composition and of the consumer product to improve the perception or enhance the performance of the benefit agent in the consumer product.

### BACKGROUND OF THE INVENTION

It is a major challenge to develop consumer products such as household care, personal care and fabric care products which provide an optimum of exposure of receptive site to effective levels of benefit agent over a long period of time. Several studies have shown that benefit agents are perceived as being more effective if they are available at an individually tailored level at a particular time at the target site. This challenge can be addressed by utilizing encapsulated benefit agent.

It is known to incorporate encapsulated benefit agents in consumer products, such as household care, personal care, fabric care and pet care products. Benefit agents include for example fragrances, cosmetic agents, food ingredients, nutraceuticals, drugs and substrate enhancers.

Benefit agents are encapsulated for a variety of reasons. Microcapsules can isolate and protect such materials from external suspending media, such as consumer product bases, in which they may be incompatible or unstable. They are also used to assist in the deposition of benefit agents onto substrates, such as skin or hair, fabrics or hard household surfaces. They can also act as a means of controlling the spatio-temporal release of a benefit agent.

Fragrance is a significant part of consumer products. Fragrances are volatile. They react with other components and are susceptible to heat, moisture and various other factors. Therefore, it is important to control the release of fragrance in the consumer product over a desired site and at a desired rate.

Scent boosters constitute a category of consumer products used to deliver a fragrance at some point of time in an application of the consumer product. Scent boosters are available in either solid or liquid forms.

Typically, a solid scent booster is added directly to a washing machine prior to starting a wash cycle. The function of a scent booster is to provide to the consumer an enhanced perception of the fragrance throughout the washing and rinsing cycles, at the moment the laundry is taken out of the machine, during drying and after the laundry has been dried.

Such a release profile is achieved by combining free, non-encapsulated fragrance components with encapsulated fragrance components, wherein the free fragrance components contribute essentially to enhancing fragrance perception on wet fabrics, while the encapsulated components contribute essentially to enhancing fragrance perception on dry fabrics. Additionally, the encapsulated components may be released during fabric handling, typically under the action of mechanical forces. Core-shell microcapsules may be used, wherein the core comprises the encapsulated fragrance components and is surrounded by an impervious, frangible shell. Both the free fragrance components and the encapsulated fragrance components are dispersed in the scent booster.

The “clean label” concept is one of the biggest trends of the decade. The term itself has many definitions including sustainable, naturally sourced and biodegradable ingredients as well as minimal processing and impact on the environment. Consumers are increasingly concerned about the sustainability of the products they use, however, in general, biodegradable and/or naturally sourced materials do not perform at the same level as their more established, non-biodegradable equivalents, therefore failing to meet the consumer's expectations.

WO 2011/056938 discloses a laundry scent additive composition in a pastille formulation containing both fragrance encapsulated in core-shell microcapsules and free fragrance dispersed within a matrix of polyethylene glycol.

US 2017/226690 discloses a scent booster composition in a pastille formulation containing both fragrance encapsulated in core-shell microcapsules and free fragrance dispersed in a matrix of clay and polyethylene glycol.

However, neither clay not polyethylene glycol is biodegradable. There is a need, therefore, to provide solid scent boosters that are biodegradable. There is also a need to provide scent boosters that are bio-based.

### SUMMARY OF THE INVENTION

In a first aspect, the present invention provides a solid composition consisting of:
a) a solid, water-soluble, biodegradable carrier; and
b) a microcapsule composition comprising a polymer encapsulating a benefit agent, wherein the benefit agent is encapsulated in core-shell microcapsules comprising a core and a shell surrounding the core;

In a further aspect, the invention provides methods of preparing the composition as described herein.

The invention further provides a consumer product comprising the composition as described herein.

The use of the solid composition and consumer product as described herein to improve the perception or enhance the performance of the benefit agent in the consumer product is provided in a further aspect.

### DEFINITIONS

The term “benefit agent” refers to any substance which, when added to a product, may improve the perception of this product by a consumer or may enhance the action of this product in an application. Examples of benefit agents include perfume or fragrance ingredients, cosmetic ingredients, bioactive agents (such as bactericides, insect repellents and pheromones), substrate enhancers (such as silicones and brighteners), enzymes (such as lipases and proteases), dyes, pigments and nutraceuticals.

The term “bio-based” relates to the origin of a material and refers to materials intentionally made from substances derived from living (or once-living) organisms, as opposed to petroleum-derived materials. The definition includes both natural materials, such as naturally-extracted proteins and polysaccharides, and materials that have undergone some degree of processing, such as cellulose fibers.

“Biodegradable” materials are defined as materials whose physical and chemical properties undergo deterioration and completely degrade when exposed to the environment. This property, therefore, relates to the end-of-life of the material. Bio-based materials can be biodegradable or non-degradable. Similarly, while many bio-based materials are biodegradable (e.g., starch), not all biodegradable materials are bio-based.

In the context of the present invention, a “biodegradable” ingredient is an ingredient which meets the pass criteria for “inherently biodegradable” and/or “readily biodegradable” in at least one OECD biodegradation study. In order to avoid any ambiguity, this means that if an ingredient passes one test but fails one or more other ones, the pass result overrules the other test results.

For assessment of the pass criteria for “readily biodegradable”, the biodegradation study can be carried out using standardised methods such as OECD Method 301C, OECD Method 301D, OECD Method 301F and OECD Method 310.

OECD Method 301C, OECD Method 301D and OECD Method 301F are described in the OECD Guidelines for the Testing of Chemicals, Section 3, Test No. 301: Ready Biodegradability (Adopted: 17 Jul. 1992; https://doi.org/10.1787/9789264070349-en).

OECD Method 310 is described in the OECD Guidelines for the Testing of Chemicals, Section 3, Test No. 310: Ready Biodegradability—CO2 in sealed vessels (Headspace Test) (Adopted: 23 Mar. 2006; Corrected: 26 Sep. 2014; https://doi.org/10.1787/9789264016316-en).

In the context of the present invention, the pass criteria for “readily biodegradable” are assessed according to OECD Method 301F, which refers to manometric respirometry. In this method the pass level for “ready biodegradability” is to reach 60% of theoretical oxygen demand and/or chemical oxygen demand. This pass value has to be reached in a 10-day window within the 28-day period of the test. The 10-day window begins when the degree of biodegradation has reached 10% of theoretical oxygen demand and/or chemical oxygen demand and must end before day 28 of the test. Given a positive result in a test of ready biodegradability, it may be assumed that the chemical will undergo rapid and ultimate biodegradation in the environment (Introduction to the OECD Guidelines for the Testing of Chemicals, Section 3, Part 1: Principles and Strategies Related to the Testing of Degradation of Organic Chemicals; Adopted: July 2003).

The term “solid” indicates that the material is in a solid state of aggregation at a temperature below about 40° C.

The term “water-soluble” indicates that the material completely dissolves in water at a temperature above about 10° C.

In the context of the present invention, all percentages refer to weight percentages (% w/w), unless otherwise indicated.

### DETAILED DESCRIPTION

Preferred and/or optional features of the invention will now be set out. Any aspect of the invention may be combined with any other aspect of the invention unless the context demands otherwise. Any of the preferred or optional features of any aspect may be combined, singly or in combination, with any aspect of the invention, as well as with any other preferred or optional features, unless the context demands otherwise.

The applicant has surprisingly and unexpectedly found that a solid composition consisting of
a) a solid, water-soluble, biodegradable carrier; and
b) a microcapsule composition comprising a polymer encapsulating a benefit agent, wherein the benefit agent is encapsulated in core-shell microcapsules comprising a core and a shell surrounding the core.
   is capable of enhancing the overall fragrance perception on a fabric compared to a conventional, non-biodegradable composition.

The invention, therefore, provides a solid composition consisting of:
a) a solid, water-soluble, biodegradable carrier; and
b) a microcapsule composition comprising a polymer encapsulating a benefit agent, wherein the benefit agent is encapsulated in core-shell microcapsules comprising a core and a shell surrounding the core.

### Carrier

The carrier employed in the present invention is solid, water-soluble and biodegradable.

Suitable carriers for the present invention include carbohydrates such as sucrose, mono-, di-, and polysaccharides and derivatives such as starch, cellulose, methyl cellulose, ethyl cellulose, propyl cellulose; hydrogenated carbohydrates; hydrolyzed carbohydrates; polyols such as threitol, arabitol, ribitol, galactitol, fucitol, iditol, inositol, sorbitol, mannitol, maltitol, lactitol, isomalt, xylitol and erythritol; or combinations thereof.

In one embodiment, the carrier is not polymeric.

The solid composition does not comprise any water soluble polymers such as starch, modified starch, maltodextrins, polysaccharides, carbohydrates, chitosan, gum arabic, polyethylene glycol (PEG), polyvinyl pyrrolidone (PVP), polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, and mixtures thereof.

In one embodiment, the carrier is a sugar alcohol. Sugar alcohols (also called polyhydric alcohols, polyalcohols, alditols or glycitols) are organic compounds, typically derived from sugars, containing one hydroxyl group (—OH) attached to each carbon atom. Since they contain multiple —OH groups, they are classified as polyols. They are white, water-soluble solids that can occur naturally or be produced industrially by hydrogenation of sugars. As such, sugar alcohols are considered as bio-based ingredients, thereby making the compositions more sustainable also from the point of view of sourcing the ingredients.

Sugar alcohols have the general formula (CHOH)ₙH₂ and within the same chemical formula they are further differentiated by the relative orientation (stereochemistry) of these —OH groups.

In one embodiment, the sugar alcohol has the general formula (CHOH)ₙH₂, wherein n is 4, 5 or 6, such as xylitol, sorbitol, arabitol, mannitol, erythritol or a mixture thereof. Optionally, the sugar alcohol is xylitol or sorbitol.

In one embodiment, the melting point of the carrier is between about 70° C. to about 200° C., optionally between 80° C. to about 150° C. The lower the melting point of the carrier, the more sustainable the composition.

In one embodiment, the proportion of the carrier is between about 70 wt.-% to about 99 wt.-%, preferably about 80 wt.-% to about 98.5 wt.-%, even more preferably about 85 wt.-% to about 98 wt.-%, relative to the total weight of the solid composition.

The hygroscopicity of different materials varies with relative humidity. Moisture pick-up will limit the shelf life of the consumer product in which the material is a major component. In one embodiment, therefore, the carrier is not hygroscopic. The advantage of such a carrier is that the solid composition will not absorb moisture during storage.

### Benefit Agent

Suitable benefit agents to be incorporated into the core of the core-shell microcapsules of the present invention include perfume ingredients, cosmetic ingredients, bioactive agents (such as bactericides, insect repellents and pheromones), substrate enhancers (such as silicones and brighteners), enzymes (such as lipases and proteases), dyes, pigments and nutraceuticals

In one embodiment, the benefit agent comprises, optionally consist of at least one fragrance ingredient. A comprehensive list of fragrance ingredients that may be encapsulated in accordance with the present invention may be found in the perfumery literature, for example “Perfume & Flavor Chemicals”, S. Arctander (Allured Publishing, 1994). Encapsulated fragrance ingredients according to the present invention preferably comprise fragrance ingredients selected from the group consisting of ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE C 8 OCTYLIC (octanal); ALDEHYDE C 9 ISONONYLIC (3,5,5-trimethylhexanal); ALDEHYDE C 9 NONYLIC FOOD GRADE (nonanal); ALDEHYDE C 90 NONENYLIC ((E)-non-2-enal); ALDEHYDE ISO C 11 ((E)-undec-9-enal); ALDEHYDE MANDARINE ((E)-dodec-2-enal); ALLYL AMYL GLYCOLATE (prop-2-enyl 2-(3-methylbutoxy)acetate); ALLYL CAPROATE (prop-2-enyl hexanoate); ALLYL CYCLOHEXYL PROPIONATE (prop-2-enyl 3-cyclohexylpropanoate); ALLYL OENANTHATE (prop-2-enyl heptanoate); AMBER CORE1-((2-(tert-butyl)cyclohexyl)oxy)butan-2-olAMBERKETAL (3,8,8, 11a-tetramethyldodecahydro-1H-3,5a-epoxynaphtho[2,1-c]oxepine); AMBERMAX (1,3,4,5,6,7-hexahydro-. beta., 1,1,5,5-pentamethyl-2H-2,4a-Methanonaphthalene-8-ethanol); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL BUTYRATE (pentyl butanoate); AMYL CINNAMIC ALDEHYDE ((Z)-2-benzylideneheptanal); AMYL SALICYLATE (pentyl 2-hydroxybenzoate); ANETHOLE SYNTHETIC ((E)-1-methoxy-4-(prop-1-en-1-yl)benzene); ANISYL ACETATE (4-methoxybenzyl acetate); APHERMATE (1-(3,3-dimethylcyclohexyl)ethyl formate); AUBEPINE PARA CRESOL (4-methoxybenzaldehyde); AURANTIOL ((E)-methyl 2-((7-hydroxy-3,7-dimethyloctylidene)amino)benzoate); BELAMBRE ((1R,2S,4R)-2′-isopropyl-1,7,7-trimethylspiro[bicyclo[2.2.1]heptane-2,4′-[1,3]dioxane]); BENZALDEHYDE (benzaldehyde); BENZYL ACETATE (benzyl acetate); BENZYL ACETONE (4-phenylbutan-2-one); BENZYL BENZOATE (benzyl benzoate); BENZYL SALICYLATE (benzyl 2-hydroxybenzoate); BERRYFLOR (ethyl 6-acetoxyhexanoate); BICYCLO NONALACTONE (octahydro-2H-chromen-2-one); BOISAMBRENE FORTE ((ethoxymethoxy)cyclododecane); BOISIRIS ((1S,2R,5R)-2-ethoxy-2,6,6-trimethyl-9-methylenebicyclo[3.3.1]nonane); BORNEOL CRYSTALS ((1S,2S,4S)-1,7,7-trimethylbicyclo[2.2.1]heptan-2-ol); BORNYL ACETATE ((2S,4S)-1,7,7-trimethylbicyclo[2.2.1]heptan-2-yl acetate); BOURGEONAL (3-(4-(tert-butyl)phenyl)propanal); BUTYL BUTYRO LACTATE (1-butoxy-1-oxopropan-2-yl butanoate); BUTYL CYCLOHEXYL ACETATE PARA (4-(tert-butyl)cyclohexyl acetate); BUTYL QUINOLINE SECONDARY (2-(2-methylpropyl)quinoline); CAMPHOR SYNTHETIC ((1S,4S)-1,7,7-trimethylbicyclo[2.2.1]heptan-2-one); CARVACROL (5-isopropyl-2-methylphenol); CARVONE LAEVO ((5R)-2-methyl-5-prop-1-en-2-ylcyclohex-2-en-1-one); CASHMERAN (1,1,2,3,3-pentamethyl-2,3,6,7-tetrahydro-1H-inden-4(5H)-one); CASSYRANE (5-tert-butyl-2-methyl-5-propyl-2H-furan); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CETONE V ((E)-1-(2,6,6-trimethylcyclohex-2-en-1-yl)hepta-1,6-dien-3-one); CINNAMIC ALCOHOL SYNTHETIC ((E)-3-phenylprop-2-en-1-ol); CINNAMIC ALDEHYDE ((2E)-3-phenylprop-2-enal); CINNAMYL ACETATE ((E)-3-phenylprop-2-en-1-yl acetate); CIS JASMONE ((Z)-3-methyl-2-(pent-2-en-1-yl)cyclopent-2-enone); CIS-3-HEXENOL ((Z)-hex-3-en-1-ol); CITRAL TECH ((E)-3,7-dimethylocta-2,6-dienal); CITRATHAL R ((Z)-1,1-diethoxy-3,7-dimethylocta-2,6-diene); CITRONELLAL (3,7-dimethyloct-6-enal); CITRONELLOL EXTRA (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CLONAL (dodecanenitrile); CORANOL (4-cyclohexyl-2-methylbutan-2-ol); COSMONE ((Z)-3-methylcyclotetradec-5-enone); COUMARIN PURE CRYSTALS (2H-chromen-2-one); CRESYL ACETATE PARA ((4-methylphenyl)acetate); CRESYL METHYL ETHER PARA (1-methoxy-4-methylbenzene); CUMIN NITRILE (4-isopropylbenzonitrile); CYCLAL C (2,4-dimethylcyclohex-3-ene-1-carbaldehyde); CYCLAMEN ALDEHYDE EXTRA (3-(4-isopropylphenyl)-2-methylpropanal); CYCLOGALBANATE (allyl 2-(cyclohexyloxy)acetate); CYCLOHEXYL ETHYL ACETATE (2-cyclohexylethyl acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); CYMENE PARA (1-methyl-4-propan-2-ylbenzene); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA (1-(2,6,6-trimethyl-1-cyclohex-3-enyl)but-2-en-1-one); DECALACTONE GAMMA (5-hexyloxolan-2-one); DECENAL-4-TRANS ((E)-dec-4-enal); DELPHONE (2-pentylcyclopentanone); DELTA-3 CARENE ((1S,6S)-3,7,7-trimethylbicyclo[4.1.0]hept-3-ene); DIHEXYL FUMARATE (dihexyl-but-2-enedioate); DIHYDRO ANETHOLE (1-methoxy-4-propylbenzene); DIHYDRO JASMONE (3-methyl-2-pentylcyclopent-2-enone); DIHYDRO MYRCENOL (2,6-dimethyloct-7-en-2-ol); DIMETHYL ANTHRANILATE (methyl 2-(methylamino)benzoate); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DIPHENYL OXIDE (oxydibenzene); DODECALACTONE DELTA (6-heptyltetrahydro-2H-pyran-2-one); DODECALACTONE GAMMA (5-octyloxolan-2-one); DODECENAL ((E)-dodec-2-enal); DUPICAL ((E)-4-((3aS,7aS)-hexahydro-1H-4,7-methanoinden-5 (6H)-ylidene)butanal); EBANOL ((E)-3-methyl-5-(2,2,3-trimethylcyclopent-3-en-1-yl)pent-4-en-2-ol); ESTERLY (ethyl cyclohexyl carboxylate); ETHYL ACETATE (ethyl acetate); ETHYL ACETOACETATE (ethyl 3-oxobutanoate); ETHYL CINNAMATE (ethyl 3-phenylprop-2-enoate); ETHYL HEXANOATE (ethyl hexanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL MALTOL (2-ethyl-3-hydroxy-4H-pyran-4-one); ETHYL METHYL-2-BUTYRATE (ethyl 2-methylbutanoate); ETHYL OCTANOATE (ethyl octanoate); ETHYL OENANTHATE (ethyl heptanoate); ETHYL PHENYL GLYCIDATE (ethyl 3-20) phenyloxirane-2-carboxylate); ETHYL SAFRANATE (ethyl 2,6,6-trimethylcyclohexa-1,3-diene-1-carboxylate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FENNALDEHYDE (3-(4-methoxyphenyl)-2-methylpropanal); FIXAMBRENE (3a,6,6,9a-tetramethyldodecahydronaphtho[2,1-b]furan); FIXOLIDE (1-(3,5,5,6,8,8-hexamethyl-5,6,7,8-tetrahydronaphthalen-2-yl)ethanone); FLORALOZONE (3-(4-ethylphenyl)-2,2-dimethylpropanal); FLORHYDRAL (3-(3-isopropylphenyl)butanal); FLORIDILE ((E)-undec-9-enenitrile); FLOROCYCLENE ((3aR,6S,7aS)-3a,4,5,6,7,7a-hexahydro-1H-4,7-methanoinden-6-yl propanoate); FLOROPAL (2,4,6-trimethyl-4-phenyl-1,3-dioxane); FLOROSA HC (tetrahydro-4-methyl-2-(2-methylpropyl)-2H-pyran-4-ol); FRESKOMENTHE (2-(sec-butyl)cyclohexanone); FRUCTONE (ethyl 2-(2-methyl-1,3-dioxolan-2-yl)acetate); FRUITATE ((3aS,4S,7R,7aS)-ethyl octahydro-1H-4,7-methanoindene-3a-carboxylate); FRUTONILE (2-methyldecanenitrile); GALBANONE PURE (1-(5,5-dimethylcyclohex-1-en-1-yl)pent-4-en-1-one); GARDENOL (1-phenylethyl acetate); GARDOCYCLENE ((3aR,6S,7aS)-3a,4,5,6,7,7a-hexahydro-1H-4,7-methanoinden-6-yl 2-methyl propanoate); GERANIOL ((E)-3,7-dimethylocta-2,6-dien-1-ol); GERANYL ACETATE ((E)-3,7-dimethylocta-2,6-dien-1-yl acetate); GERANYL CROTONATE ((E)-3,7-dimethylocta-2,6-dien-1-yl but-2-enoate); GERANYL ISOBUTYRATE ((E)-3,7-dimethylocta-2,6-dien-1-yl 2-methylpropanoate); GIVESCONE (ethyl 2-ethyl-6,6-dimethylcyclohex-2-enecarboxylate); HABANOLIDE ((E)-oxacyclohexadec-12-en-2-one); HEDIONE (methyl 3-oxo-2-pentylcyclopentaneacetate); HELIOTROPINE CRYSTALS (benzo[d][1,3]dioxole-5-carbaldehyde); HERBANATE ((2S)-ethyl 3-isopropylbicyclo[2.2.1]hept-5-ene-2-carboxylate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS BUTYRATE ((Z)-hex-3-en-1-yl butanoate); HEXENYL-3-CIS ISOBUTYRATE ((Z)-hex-3-en-1-yl 2-methylpropanoate); HEXENYL-3-CIS SALICYLATE ((Z)-hex-3-en-1-yl 2-hydroxybenzoate); HEXYL ACETATE (hexyl acetate); HEXYL BENZOATE (hexyl benzoate); HEXYL BUTYRATE (hexyl butanoate); HEXYL CINNAMIC ALDEHYDE ((E)-2-benzylideneoctanal); HEXYL ISOBUTYRATE (hexyl 2-methylpropanoate); HEXYL SALICYLATE (hexyl 2-hydroxybenzoate); HYDROXYCITRONELLAL (7-hydroxy-3,7-dimethyloctanal); INDOFLOR (4,4a,5,9b-tetrahydroindeno[1,2-d][1,3]dioxine); INDOLE PURE (1H-indole); INDOLENE (8,8-di(1H-indol-3-yl)-2,6-dimethyloctan-2-ol); IONONE BETA ((E)-4-(2,6,6-trimethylcyclohex-1-en-1-yl)but-3-en-2-one); IRISANTHEME ((E)-3-methyl-4-(2,6,6-trimethylcyclohex-2-en-1-yl)but-3-en-2-one); IRISONE ALPHA ((E)-4-(2,6,6-trimethylcyclohex-2-en-1-yl)but-3-en-2-one); IRONE ALPHA ((E)-4-(2,5,6,6-tetramethylcyclohex-2-en-1-yl)but-3-en-2-one); ISO E SUPER (1-(2,3,8,8-tetramethyl-1,2,3,4,5,6,7,8-octahydronaphthalen-2-yl)ethanone); ISOAMYL ACETATE (3-methylbutyl acetate); ISOAMYL BUTYRATE (3-methylbutyl butanoate); ISOBUTYL METHOXY PYRAZINE (2-methylpropyl 3-methoxypyrazine); ISOCYCLOCITRAL (2,4,6-trimethylcyclohex-3-enecarbaldehyde); ISOEUGENOL ((E)-2-methoxy-4-(prop-1-en-1-yl)phenol); ISOJASMONE B 11 (2-hexylcyclopent-2-en-1-one); ISOMENTHONE DL (2-isopropyl-5-methylcyclohexanone); ISONONYL ACETATE (3,5,5-trimethylhexyl acetate); ISOPROPYL METHYL-2-BUTYRATE (isopropyl 2-methylbutanoate); ISOPROPYL QUINOLINE (6-isopropylquinoline); ISORALDEINE ((E)-3-methyl-4-(2,6,6-trimethylcyclohex-2-en-1-yl)but-3-en-2-one); JASMACYCLENE ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); JASMONYL (3-butyl-5-methyltetrahydro-2H-pyran-4-yl acetate); JASMOPYRANE FORTE (3-pentyltetrahydro-2H-pyran-4-yl acetate); JAVANOL ((1-methyl-2-((1,2,2-trimethylbicyclo[3.1.0]hexan-3-yl)methyl)cyclopropyl)methanol); KOAVONE ((Z)-3,4,5,6,6-pentamethylhept-3-en-2-one); LAITONE (8-isopropyl-1-oxaspiro[4.5]decan-2-one); LEAF ACETAL ((Z)-1-(1-ethoxyethoxy)hex-3-ene); LEMONILE ((2E,6Z)-3,7-dimethylnona-2,6-dienenitrile); LIFFAROME ((Z)-hex-3-en-1-yl methyl carbonate); LILIAL (3-(4-(tert-butyl)phenyl)-2-methylpropanal); #N/ALINALOOL (3,7-dimethylocta-1,6-dien-3-ol); LINALOOL OXIDE (2-(5-methyl-5-vinyltetrahydrofuran-2-yl)propan-2-ol); LINALYL ACETATE (3,7-dimethylocta-1,6-dien-3-yl acetate); MAHONIAL ((4E)-9-hydroxy-5,9-dimethyl-4-decenal); MALTOL (3-hydroxy-2-methyl-4H-pyran-4-one); MALTYL ISOBUTYRATE (2-methyl-4-oxo-4H-pyran-3-yl 2-methylpropanoate); MANZANATE (ethyl 2-methylpentanoate); MAYOL ((4-isopropylcyclohexyl)methanol); MEFROSOL (3-methyl-5-phenylpentan-1-ol); MELONAL (2,6-dimethylhept-5-enal); #N/A #N/AMERCAPTO-8-METHANE-3-ONE (mercapto-para-menthan-3-one); METHYL ANTHRANILATE (methyl 2-aminobenzoate); METHYL BENZOATE (methyl benzoate); METHYL CEDRYL KETONE (1-((1S,8aS)-1,4,4,6-tetramethyl-2,3,3a,4,5,8-hexahydro-1H-5,8a-methanoazulen-7-yl)ethanone); METHYL CINNAMATE (methyl 3-phenylprop-2-enoate); METHYL DIANTILIS (2-ethoxy-4-(methoxymethyl)phenol); METHYL DIHYDRO ISOJASMONATE (methyl 2-hexyl-3-oxocyclopentane-1-carboxylate); METHYL HEPTENONE PURE (6-methylhept-5-en-2-one); METHYL LAITONE (8-methyl-1-oxaspiro[4.5]decan-2-one); METHYL NONYL KETONE (undecan-2-one); METHYL OCTYNE CARBONATE (methyl non-2-ynoate); METHYL PAMPLEMOUSSE (6,6-dimethoxy-2,5,5-trimethylhex-2-ene); METHYL SALICYLATE (methyl 2-hydroxybenzoate); MUSCENONE ((Z)-3-20) methylcyclopentadec-5-enone); MYRALDENE (4-(4-methylpent-3-en-1-yl)cyclohex-3-enecarbaldehyde); MYRCENE (7-methyl-3-methyleneocta-1,6-diene); MYSTIKAL (2-methylundecanoic acid); NECTARYL (2-(2-(4-methylcyclohex-3-en-1-yl)propyl)cyclopentanone); NEOBERGAMATE FORTE (2-methyl-6-methyleneoct-7-en-2-yl acetate); NEOCASPIRENE EXTRA (10-isopropyl-2,7-dimethyl-1-oxaspiro[4.5]deca-3,6-diene); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLIDYLE ((Z)-3,7, 11-trimethyldodeca-1,6, 10-trien-3-yl acetate); NEROLINE CRYSTALS (2-ethoxynaphthalene); NEROLIONE (1-(3-methylbenzofuran-2-yl)ethanone); NERYL ACETATE ((Z)-3,7-dimethylocta-2,6-dien-1-yl acetate); NIRVANOLIDE ((E)-13-methyloxacyclopentadec-10-en-2-one); NONADIENAL ((2E,6Z)-nona-2,6-dienal); NONADIENOL-2,6 ((2Z,6E)-2,6-nonadien-1-ol); NONADYL (6,8-dimethylnonan-2-ol); NONALACTONE GAMMA (5-pentyloxolan-2-one); NONENAL-6-CIS ((Z)-non-6-enal); NONENOL-6-CIS ((Z)-non-6-en-1-ol); NOPYL ACETATE (2-(6,6-dimethylbicyclo[3.1.1]hept-2-en-2-yl)ethyl acetate); NYMPHEAL (3-(4-(2-methylpropyl)-2-methylphenyl)propanal); OCTALACTONE DELTA (6-propyltetrahydro-2H-pyran-2-one); METHYL HEXYL KETONE (octan-2-one); ORANGER CRYSTALS (1-(2-naphtalenyl)-ethanone); ORIVONE (4-(tert-pentyl)cyclohexanone); PANDANOL ((2-methoxyethyl)benzene); PARA TERT BUTYL CYCLOHEXYL ACETATE (4-(tert-butyl)cyclohexyl acetate); PARADISAMIDE (2-ethyl-N-methyl-N-(m-tolyl)butanamide); PEACH PURE (5-heptyldihydrofuran-2 (3H)-one); PELARGENE (2-methyl-4-methylene-6-phenyltetrahydro-2H-pyran); PELARGOL (3,7-dimethyloctan-1-ol); PEONILE (2-cyclohexylidene-2-phenylacetonitrile); PETALIA (2-cyclohexylidene-2-(o-tolyl)acetonitrile); PHARAONE (2-cyclohexylhepta-1,6-dien-3-one); PHENOXY ETHYL ISOBUTYRATE (2-(phenoxy)ethyl 2-methylpropanoate); PHENYL ACETALDEHYDE (2-phenyl-ethanal); PHENYL ETHYL ACETATE (2-phenylethyl acetate); PHENYL ETHYL ALCOHOL (2-phenylethanol); PHENYL ETHYL ISOBUTYRATE (2-phenylethyl 2-methylpropanoate); PHENYL ETHYL PHENYL ACETATE (2-phenylethyl 2-phenylacetate); PHENYL PROPYL ALCOHOL (3-phenylpropan-1-ol); PINENE ALPHA (2,6,6-trimethylbicyclo[3.1.1]hept-2-ene); PINENE BETA (6,6-dimethyl-2-methylenebicyclo[3.1.1]heptane); PINOACETALDEHYDE (3-(6,6-dimethylbicyclo[3.1.1]hept-2-en-2-yl)propanal); PIVAROSE (2,2-dimethyl-2-pheylethyl propanoate); POMAROSE ((2E,5E)-5,6,7-trimethylocta-2,5-dien-4-one); POMELOL (2,4,7-Trimethyl-6-octen-1-ol); PRECYCLEMONE B (1-methyl-4-(4-methylpent-3-en-1-yl)cyclohex-3-enecarbaldehyde); PRENYL ACETATE (3-methylbut-2-en-1-yl acetate); PRUNOLIDE (5-pentyldihydrofuran-2(3H)-one); RADJANOL SUPER ((E)-2-ethyl-4-(2,2,3-trimethylcyclopent-3-en-1-yl)but-2-en-1-ol); RASPBERRY KETONE (4-(4-hydroxyphenyl)butan-2-one); RHUBAFURAN (2,4-dimethyl-4-phenyltetrahydrofuran); ROSACETOL (2,2,2-trichloro-1-phenylethyl acetate); ROSALVA (dec-9-en-1-ol); ROSE OXIDE (4-methyl-2-(2-methylprop-1-en-1-yl)tetrahydro-2H-pyran); ROSE OXIDE CO (4-methyl-2-(2-methylprop-1-en-1-yl)tetrahydro-2H-pyran); ROSYFOLIA (1-methyl-2-(5-methylhex-4-en-2-yl)cyclopropylmethanol); ROSYRANE SUPER (4-methyl-2-phenyl-3,6-dihydro-2H-pyran); SAFRALEINE (2,3,3-trimethyl-1-indanone); SAFRANAL (2,6,6-trimethylcyclohexa-1,3-dienecarbaldehyde); SANDALORE EXTRA (3-methyl-5-(2,2,3-trimethylcyclopent-3-en-1-yl)pentan-2-ol); SCENTAURUS CLEAN (ethyl (Z)-2-acetyl-4-methyltridec-2-enoate); SCENTAURUS JUICY (4-(dodecylthio)-4-methylpentan-2-one); SERENOLIDE (2-(1-(3,3-dimethylcyclohexyl)ethoxy)-2-methylpropyl cyclopropanecarboxylate); SILVANONE SUPRA (cyclopentadecanone, hexadecanolide); SILVIAL (2-methyl-3-[4-(2-methylpropyl)phenyl]propanal); SPIROGALBANONE (1-(spiro[4.5]dec-6-en-7-yl)pent-4-en-1-one); STEMONE ((E)-5-methylheptan-3-one oxime); STYRALLYL ACETATE (1-phenylethyl acetate); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclopropanecarboxylate); TERPINENE ALPHA (1-methyl-4-propan-2-ylcyclohexa-1,3-diene); TERPINENE GAMMA (1-methyl-4-propan-2-ylcyclohexa-1,4-diene); TERPINEOL (2-(4-methylcyclohex-3-en-1-yl)propan-2-ol); TERPINEOL ALPHA (2-(4-methyl-1-cyclohex-3-enyl)propan-2-ol); TERPINEOL PURE (2-(4-methylcyclohex-3-en-1-yl)propan-2-ol); TERPINOLENE (1-methyl-4-(propan-2-ylidene)cyclohex-1-ene); TERPINYL ACETATE (2-(4-methyl-1-cyclohex-3-enyl)propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); THYMOL (2-isopropyl-5-methylphenol); TOSCANOL (1-(cyclopropylmethyl)-4-methoxybenzene); TRICYCLAL (2,4-dimethylcyclohex-3-enecarbaldehyde); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); TROPIONAL (3-(benzo[d][1,3]dioxol-5-yl)-2-methylpropanal); UNDECATRIENE ((3E,5Z)-undeca-1,3,5-triene); UNDECAVERTOL ((E)-4-methyldec-3-en-5-ol); VANILLIN (4-hydroxy-3-methoxybenzaldehyde); VELOUTONE (2,2,5-trimethyl-5-pentylcyclopentanone); VELVIONE ((Z)-cyclohexadec-5-enone); VIOLET NITRILE ((2E,6Z)-nona-2,6-dienenitrile); YARA YARA (2-methoxynaphtalene); ZINARINE (2-(2,4-dimethylcyclohexyl)pyridine; BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These fragrance ingredients are particularly suitable for obtaining stable and performing microcapsules, owing to their favorable lipophilicity and olfactive performance.

In one embodiment of the present invention, more than 75%, preferably more than 80%, even more preferably more than 85%, even still more preferably more than 90%, even yet still more preferably more than 95%, of the fragrance ingredients are biodegradable and selected from ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA (2-methylundecanal); ALDEHYDE C 8 OCTYLIC (octanal); CYCLAMEN ALDEHYDE EXTRA (3-(4-isopropylphenyl)-2-methylpropanal); ALDEHYDE ISO C 11 ((E)-undec-9-enal); ALLYL AMYL GLYCOLATE (prop-2-enyl 2-(3-methylbutoxy)acetate); ALLYL CYCLOHEXYL PROPIONATE (prop-2-enyl 3-cyclohexylpropanoate); ALLYL OENANTHATE (prop-2-enyl heptanoate); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL SALICYLATE (pentyl 2-hydroxybenzoate); AUBEPINE PARA CRESOL (4-methoxybenzaldehyde); BENZYL ACETATE (benzyl acetate); BENZYL SALICYLATE (benzyl 2-hydroxybenzoate); BORNYL ACETATE ((2S,4S)-1,7,7-trimethylbicyclo[2.2.1]heptan-2-yl acetate); CARVACROL (5-isopropyl-2-methylphenol); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CITRAL ((E)-3,7-dimethylocta-2,6-dienal); CITRONELLOL (3,7-dimethyloct-6-en-1-ol); CITRONELLYL ACETATE (3,7-dimethyloct-6-en-1-yl acetate); COSMONE ((Z)-3-methylcyclotetradec-5-enone); CRESYL METHYL ETHER PARA (1-methoxy-4-methylbenzene); CYCLOHEXYL ETHYL ACETATE (2-cyclohexylethyl acetate); CYCLOHEXYL SALICYLATE (cyclohexyl 2-hydroxybenzoate); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DECALACTONE GAMMA (5-hexyloxolan-2-one); DECENAL-4-TRANS ((E)-dec-4-enal); DIHYDRO MYRCENOL (2,6-dimethyloct-7-en-2-ol); DIPHENYL OXIDE (oxydibenzene); DIHYDRO ANETHOLE (1-methoxy-4-propylbenzene); DIHYDRO JASMONE (3-methyl-2-pentylcyclopent-2-enone); DIMETHYL ANTHRANILATE (methyl 2-(methylamino)benzoate); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETOL (2,6-dimethylheptan-2-ol); DODECALACTONE DELTA (6-heptyltetrahydro-2H-pyran-2-one); DODECALACTONE GAMMA (5-octyloxolan-2-one); DODECENAL ((E)-dodec-2-enal); EBANOL ((E)-3-methyl-5-(2,2,3-trimethylcyclopent-3-en-1-yl)pent-4-en-2-ol); ETHYL HEXANOATE (ethyl hexanoate); ETHYL METHYL-2-BUTYRATE (ethyl 2-methyl butyrate); ETHYL MALTOL (2-ethyl-3-hydroxy-4H-pyran-4-one); ETHYL OENANTHATE (ethyl heptanoate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FIXAMBRENE (3a,6,6,9a-tetramethyldodecahydronaphtho[2,1-b]furan); FLORHYDRAL (3-(3-isopropylphenyl)butanal); FLORIDILE ((E)-undec-9-enenitrile); GALBANONE PURE (1-(5,5-dimethylcyclohex-1-en-1-yl)pent-4-en-1-one); GARDENOL (1-phenylethyl acetate); GERANIOL ((E)-3,7-dimethylocta-2,6-dien-1-ol); GERANYL ACETATE ((E)-3,7-dimethylocta-2,6-dien-1-yl acetate); HABANOLIDE ((E)-oxacyclohexadec-12-en-2-one); HEDIONE (methyl 3-oxo-2-pentylcyclopentaneacetate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS SALICYLATE ((Z)-hex-3-en-1-yl 2-hydroxybenzoate); HEXYL ACETATE (hexyl acetate); INDOLENE (8,8-di(1H-indol-3-yl)-2,6-dimethyloctan-2-ol); IONONE BETA ((E)-4-(2,6,6-trimethylcyclohex-1-en-1-yl)but-3-en-2-one); IRISANTHEME ((E)-3-methyl-4-(2,6,6-trimethylcyclohex-2-en-1-yl)but-3-en-2-one); IRISONE ALPHA ((E)-4-(2,6,6-trimethylcyclohex-2-en-1-yl)but-3-en-2-one); ISOAMYL ACETATE (3-methylbutyl acetate); ISOAMYL BUTYRATE (3-methylbutyl butanoate); ISOEUGENOL ((E)-2-methoxy-4-(prop-1-en-1-yl)phenol); ISOJASMONE B 11 (2-hexylcyclopent-2-en-1-one); ISORALDEINE ((E)-3-methyl-4-(2,6,6-trimethylcyclohex-2-en-1-yl)but-3-en-2-one); JASMONYL (3-butyl-5-methyltetrahydro-2H-pyran-4-yl acetate); LAITONE (8-isopropyl-1-oxaspiro[4.5]decan-2-one); LEMONILE ((2E,6Z)-3,7-dimethylnona-2,6-dienenitrile); LINALOOL (3,7-dimethylocta-1,6-dien-3-ol); LINALOOL OXIDE (2-(5-methyl-5-vinyltetrahydrofuran-2-yl)propan-2-ol); LINALYL ACETATE (3,7-dimethylocta-1,6-dien-3-yl acetate); MANZANATE (ethyl 2-methylpentanoate); MAYOL ((4-isopropylcyclohexyl)methanol); MEFROSOL (3-methyl-5-phenylpentan-1-ol); MELONAL (2,6-dimethylhept-5-enal); MERCAPTO-8-METHANE-3-ONE (mercapto-para-menthan-3-one); METHYL ANTHRANILATE (methyl 2-aminobenzoate); METHYL BENZOATE (methyl benzoate); METHYL DIANTILIS (2-ethoxy-4-(methoxymethyl)phenol); METHYL HEPTENONE PURE (6-methylhept-5-en-2-one); METHYL LAITONE (8-methyl-1-oxaspiro[4.5]decan-2-one); METHYL OCTYNE CARBONATE (methyl non-2-ynoate); METHYL SALICYLATE (methyl 2-hydroxybenzoate); NECTARYL (2-(2-(4-methylcyclohex-3-en-1-yl)propyl)cyclopentanone); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLINE CRYSTALS (2-ethoxynaphthalene); NEROLIONE (1-(3-methylbenzofuran-2-yl)ethanone); NERYL ACETATE ((Z)-3,7-dimethylocta-2,6-dien-1-yl acetate); NONADIENAL ((2E,6Z)-nona-2,6-dienal); NONENAL-6-CIS ((Z)-non-6-enal); NONENOL-6-CIS ((Z)-non-6-en-1-ol); NYMPHEAL (3-(4-(2-methylpropyl)-2-methylphenyl)propanal); OCTALACTONE DELTA (6-propyltetrahydro-2H-pyran-2-one); ORANGER CRYSTALS (1-(2-naphtalenyl)-ethanone); PARA TERT BUTYL CYCLOHEXYL ACETATE (4-(tert-butyl)cyclohexyl acetate); PEACH PURE (5-heptyldihydrofuran-2(3H)-one); PELARGOL (3,7-dimethyloctan-1-ol); PHENYL ETHYL ACETATE (2-phenylethyl acetate); PINENE ALPHA (2,6,6-trimethylbicyclo[3.1.1]hept-2-ene); PINENE BETA (6,6-dimethyl-2-methylenebicyclo[3.1.1]heptane); POMAROSE ((2E,5E)-5,6,7-trimethylocta-2,5-dien-4-one); POMELOL FF (2,4,7-Trimethyl-6-octen-1-ol); PRENYL ACETATE (3-methylbut-2-en-1-yl acetate); PRUNOLIDE (5-pentyldihydrofuran-2 (3H)-one); RASPBERRY KETONE (4-(4-hydroxyphenyl)butan-2-one); ROSALVA (dec-9-en-1-ol); ROSE OXIDE CO (4-methyl-2-(2-methylprop-1-en-1-yl)tetrahydro-2H-pyran); ROSYRANE SUPER (4-methyl-2-phenyl-3,6-dihydro-2H-pyran); SAFRANAL (2,6,6-trimethylcyclohexa-1,3-dienecarbaldehyde); SCENTAURUS JUICY (4-(dodecylthio)-4-methylpentan-2-one); SILVIAL (2-methyl-3-[4-(2-methylpropyl)phenyl]propanal); STYRALLYL ACETATE (1-phenylethyl acetate); SYLKOLIDE ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINEOL (2-(4-methylcyclohex-3-en-1-yl)propan-2-ol); TERPINOLENE (1-methyl-4-(propan-2-ylidene)cyclohex-1-ene); TETRAHYDRO LINALOOL (3,7-dimethyloctan-3-ol); TOSCANOL (1-(cyclopropylmethyl)-4-methoxybenzene); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); UNDECAVERTOL ((E)-4-methyldec-3-en-5-ol); YARA YARA (2-methoxynaphtalene); BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil).

The above-mentioned ingredients have all been identified as not only fulfilling at least one of the aforementioned biodegradability criteria, but also as being suitable for encapsulation with respect to their physical and chemical properties, such as lipophilicity, molecular size and reactivity towards shell materials. They therefore provide a useful selection of perfume ingredients for readily and reliably providing more sustainable fragrance encapsulates.

In one embodiment, the benefit agent may comprise at least one fragrance precursor, meaning a material that is capable of releasing a fragrance ingredient by the means of a stimulus, such as a change of temperature, the presence of oxidants, the action of enzymes or the action of light. Such fragrance precursors are well-known to the art.

In one embodiment, the benefit agent may comprise at least one functional cosmetic ingredient. The functional cosmetic ingredients for use in the encapsulated composition are preferably hydrophobic. Optionally, the cosmetic ingredients have a calculated octanol/water partition coefficient (ClogP) of 1.5 or more, optionally 3 or more. Alternatively, the ClogP of the cosmetic ingredient is from 2 to 7.

Particularly useful functional cosmetic ingredients may be selected from the group consisting of emollients, smoothening ingredients, hydrating ingredients, soothing and relaxing ingredients, decorative ingredients, deodorants, anti-aging ingredients, cell rejuvenating ingredients, draining ingredients, remodeling ingredients, skin levelling ingredients, preservatives, anti-oxidants, antibacterial or bacteriostatic ingredients, cleansing ingredients, lubricating ingredients, structuring ingredients, hair conditioning ingredients, whitening ingredients, texturing ingredients, softening ingredients, anti-dandruff ingredients, and exfoliating ingredients.

Examples of suitable functional cosmetic ingredients include, but are not limited to hydrophobic polymers, such as alkyldimethylsiloxanes, polymethylsil-sesquioxanes, polyethylene, polyisobutylene, styrene-ethylene-styrene and styrene-butylene-styrene block copolymers, and the like; mineral oils, such as hydrogenated isoparaffins, silicone oils and the like; vegetable oils, such as argan oil, jojoba oil, aloe vera oil, and the like; fatty acids and fatty alcohols and their esters; glycolipides; phospholipides; sphingolipides, such as ceramides; sterols and steroids; terpenes, sesquiterpenes, triterpenes and their derivatives; essential oils, such as Arnica oil, Artemisia oil, Bark tree oil, Birch leaf oil, Calendula oil, Cinnamon oil, Echinacea oil, Eucalyptus oil, Ginseng oil, Jujube oil, Helianthus oil, Jasmine oil, Lavender oil, Lotus seed oil, Perilla oil, Rosmary oil, Sandal wood oil, Tea tree oil, Thyme oil, Valerian oil, Wormwood oil, Ylang Ylang oil, and Yucca oil.

In particular, the at least one functional cosmetic ingredient may be selected from the group consisting of Sandal wood oil, such as Fusanus Spicatus kernel oil; Panthenyl triacetate; Tocopheryl acetate; Tocopherol; Naringinin; Ethyl linoleate; Farnesyl acetate; Farnesol; Citronellyl methyl crotonate; and Ceramide-2 (1-Stearoiyl-C18-Sphingosine, CAS-No: 100403-19-8).

In one embodiment, the benefit agent may comprise agents which suppress or reduce malodour and its perception by adsorbing odour, agents which provide a warming or cooling effect, insect repellents or UV absorbers.

### Microcapsule Composition

The microcapsules of the present invention are presented in the form core-shell microcapsules, wherein the core comprising a benefit agent is encapsulated within a shell material.

Core-shell microcapsule compositions are generally provided in the form of a slurry, that is, a dispersion or suspension of microcapsules in an aqueous medium, that may contain somewhere in the order of 60 wt.-% of water. If desired, slurries can be dried to provide microcapsule compositions in the form of a powder or cake, which generally comprises around 5 wt.-% of water.

In one embodiment, the shell of the core-shell microcapsules comprises a polymer selected from the group consisting of a melamine-formaldehyde polymer, an urea-formaldehyde polymer, a polyurea, a polyurethane, a polyamide, a polyacrylate, a polycarbonate, and mixtures thereof, as defined hereinabove.

### Thermosetting Resins

Thermosetting resins are typically obtained by reacting polyfunctional monomers, such as amines, isocyanates, alcohols or phenols, chlorocarboxylic acids, (meth)acrylates, epoxides, silanes and aldehydes.

Thermosetting resins, such as aminoplast, polyurea and polyurethane resins, as well as combinations thereof are commonly employed as shell materials in the preparation of core-shell microcapsules. They are particularly valued for their resistance to leakage of the benefit agent when dispersed in aqueous suspending media, even in surfactant-containing media.

In one embodiment, the shell may comprise a melamine-formaldehyde polymer. This type of core-shell capsule has proved to be particularly suitable for benefit agent encapsulation and is described, for instance in WO 2008/098387 A1, WO 2016/207180 A1 and WO 2017/001672 A1.

In one embodiment, the shell may comprise a polyurea or polyurethane polymer. Also this type of core-shell capsule has been successfully used for benefit agent encapsulation and has the advantage to address consumer concerns with regard to residual formaldehyde in the composition. Such capsules are also described, for instance in WO 2019/174978 A1.

In one embodiment, the shell may comprise, a polyacrylate, one or more monoethylenically unsaturated and/or polyethylenically unsaturated monomer(s) in polymerized form. This type of core-shell capsule has also been successfully used for benefit agent encapsulation. Such capsules are described in the prior art, for instance in WO 2013/111912 A1 or WO 2014/032920 A1.

### Polymeric Stabiliser

The shell may comprise a polymeric stabilizer that is formed by combination of a polymeric surfactant with at least one aminosilane. The shell may further comprise a polysaccharide, preferably a polysaccharide comprising beta (1→4) linked monosaccharide units, even more preferably a cellulose derivative, in particular selected form the group consisting of hydroxyethyl cellulose, hydroxypropylmethyl cellulose, cellulose acetate and carboxymethyl cellulose, preferably hydroxyethyl cellulose.

The term “polymeric surfactant” refers to a polymer or a mixture comprising at least one polymer that has the property of lowering the interfacial tension between an oil phase and an aqueous phase, when dissolved in one or both of the phases. This ability to lower interfacial tension is called “interfacial activity”.

The term “formed by combination” in the present context means that the polymeric surfactant and the at least one aminosilane are brought in contact with each other to generate the polymeric stabilizer. Without wishing to be bound by theory, this formation can be the result of an interaction between the polymeric surfactant and the at least one aminosilane, such as through dispersion forces, electrostatic forces or hydrogen bonds. A chemical reaction, in strict sense, to form covalent bonds is also encompassed by this term.

In other words, the polymeric stabilizer can be regarded as an assembly which comprises moieties derived from a polymeric surfactant and moieties derived from at least one aminosilane.

The polymeric surfactant is soluble or dispersible in an aqueous phase or in water, respectively. This means that the individual polymeric surfactant macromolecules are substantially separated from each other in these liquids. The resulting system appears transparent or hazy when inspected by the human eye.

The polymeric stabilizer can be a relevant factor to the balance between microcapsule stability with respect to both benefit agent leakage during storage and benefit agent release under in-use conditions. In particular, the importance of providing additional stabilization of the oil-water interface has been recognized. The polymeric stabilizer thus provides a stable platform, which allows for the addition of further shell materials and/or shell precursors to form novel encapsulated benefit agent compositions. More specifically, the addition of a polysaccharide, preferably a polysaccharide comprising beta (1→4) linked monosaccharide units, even more preferably a cellulose derivative, leads to highly sustainable microcapsules with an excellent release profile.

The polysaccharide may be deposited on the outer surface of the capsule shell formed by the polymeric stabilizer. This results in a multilayer shell having at least one layer of polymeric stabilizer and one layer of polysaccharide. It may improve the imperviousness of the encapsulating shell by increasing the amount of encapsulating material.

To avoid any ambiguity, the present invention is not restricted to a shell having sharply defined discrete layers, although this is one possible embodiment. More specifically, the layers can also be gradual and undiscrete. On the other hand, and at the other extreme, the shell can even be essentially homogenous.

The polysaccharide may react with unreacted groups of the polymeric stabilizer and increase the density of the cross-linked shell. The polysaccharide may also interact with the polymeric stabilizer by physical forces, physical interactions, such as hydrogen bonding, ionic interactions, hydrophobic interactions or electron transfer interactions.

The shell additionally comprising a polysaccharide can be further stabilized with a stabilizing agent. Preferably the stabilizing agent comprises at least two carboxylic acid groups. Even more preferably, the stabilizing agent is selected from the group consisting of citric acid, benzene-1,3,5-tricarboxylic acid, 2,5-furandicarboxylic acid, itaconic acid, poly(itaconic acid) and combinations thereof.

In a particular embodiment of the present invention, the polymeric surfactant comprises, in particular consists of, a polysaccharide comprising carboxylate groups. It has been found that combining such a polymeric surfactant with at least one aminosilane results in the formation of a polymeric stabilizer, which is more sustainable than stabilizers known in the prior art, particular in terms of environment and resources protection. Without wishing to be bound by theory, it is believed that the carboxylate groups may interact with the at least one aminosilane in a manner mentioned hereinabove.

The polysaccharide comprising carboxylate groups may comprise uronic acid units, in particular hexuronic acid units. Polysaccharides having uronic acid units, in particular hexuronic acid units, are broadly available in nature.

The hexuronic acid units can be selected from the group consisting of galacturonic acid units, glucuronic acid units, in particular 4-O-methyl-glucuronic acid units, guluronic acid units and mannuronic acid units.

The polysaccharide comprising carboxylate groups may be branched. Branched polysaccharides comprising carboxylate groups have the advantage of forming more compact networks than linear polysaccharides and therefore may favor the imperviousness of the encapsulating shell, resulting in reduced leakage and greater encapsulation efficiency.

The polymeric surfactant can be selected from pectin, gum Arabic and an alginate. These polysaccharides offer a most suitable combination of solubility, viscosity and interfacial activity that make the microcapsules particularly performing in terms of handling, storage stability and olfactive performance. The polymeric surfactant may also be hyaluronic acid.

The carboxylate groups can be at least partially present in the form of the corresponding carboxylate salt, in particular the corresponding sodium, potassium, magnesium or calcium carboxylate salt.

In particular embodiments of the present invention, the polyanion is selected from the group consisting of pectin, gum arabic and alginate.

Among the pectins, the carboxylate groups can be partially present in the form of the corresponding methyl ester. The percentage of carboxylate groups that are present in the form of the corresponding methyl ester can be from 3% to 95%, preferably from 4% to 75%, more preferably from 5 to 50%. Pectins comprising carboxylic groups, of which 50% or more are present in the form of the corresponding methyl ester, are referred to as “high methoxylated”. Pectins comprising carboxylate groups, of which less than 50% are present in the form of the corresponding methyl ester, are referred to as “low methoxylated”.

Among the two variants of gum Arabic, i.e. gum acacia Senegal and gum acacia Seyal, gum acacia Senegal is preferred, owing to the higher level of glucuronic acid in gum acacia Senegal.

The aminosilane employed in the formation of the polymeric stabilizer can be selected from a compound of Formula (I).

Si(R¹)(R²)(OR³)_{(3-f)} Formula (I)
wherein R¹ is a linear or branched alkyl or alkenyl residue comprising an amine functional group; R² is each independently a linear or branched alkyl group with 1 to 4 carbon atoms; R³ is each independently a H or a linear or branched alkyl group with 1 to 4 carbon atoms; and f is 0, 1 or 2.

The silane groups may undergo polycondensation reactions with one another to form a silica network at the oil/water interface that additionally stabilizes this interface.

In one embodiment, R² and R³ are each independently methyl or ethyl.

In one embodiment, f is 0 or 1.

In one embodiment, R¹ is a C₁-C₁₂ linear or branched alkyl or alkenyl residue comprising an amine functional group. Optionally, R¹ is a C₁-C₄ linear or branched alkyl or alkenyl residue comprising an amine functional group.

In one embodiment, the amine functional group is a primary, a secondary or a tertiary amine.

In one embodiment, the at least one aminosilane is a bipodal aminosilane. By “bipodal aminosilane” it is meant a molecule comprising at least one amino group and two residues, each of these residues bearing at least one alkoxysilane moiety. Bipodal aminosilanes are particularly advantageous for forming stable oil-water interfaces, compared to conventional aminosilanes. Without wishing to be bound by theory, it is believed that this beneficial role is due to the particular, bi-directional arrangement of the silane moieties in the molecule of a bipodal aminosilane, which allows formation of a more tightly linked silica network at the oil-water interface.

In one embodiment, the bipodal aminosilane is a compound of Formula (II).

(O—R³)_{(3-f)}(R²)_{f}Si—R⁴—X—R⁴—Si(O—R³)_{(3-f)}(R²)_{f} Formula (II)
wherein X is —NR⁵—, —NR⁵—CH₂—NR⁵—, —NR⁵—CH₂—CH₂—NR⁵—, —NR⁵—CO—NR⁵—, or

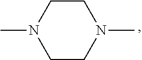

R² is each independently a linear or branched alkyl group with 1 to 4 carbon atoms;
R³ is each independently H or a linear or branched alkyl group with 1 to 4 carbon atoms;
R⁴ is each independently a linear or branched alkylene group with 1 to 6 carbon atoms;
R⁵ is each independently H, CH₃ or C₂H₅; and
f is each independently 0, 1 or 2.

In one embodiment, R² is CH₃ or C₂H₅.

In one embodiment, R³ is CH₃ or C₂H₅.

In one embodiment, R⁴ is —CH₂—, —CH₂—CH₂— or —CH₂—CH₂—CH₂—.

In one embodiment, R⁵ is H or CH₃.

In one embodiment, f is 0 or 1.

Examples of suitable bipodal aminosilanes include, but are not limited to, bis(3-(triethoxysilyl)propyl)amine, N,N′-bis(3-(trimethoxysilyl)propyl)urea, bis(3-(methyldiethoxysilyl) propyl)amine, N,N′-bis(3-(trimethoxysilyl)propyl)ethane-1,2-diamine, bis(3-(methyldimethoxysilyl)propyl)-N-methylamine and N,N′-bis(3-(triethoxysilyl)propyl)piperazine.

In one embodiment, the bipodal aminosilane is bis(3-(triethoxysilyl)propyl)amine, which has the advantage of releasing ethanol instead of more toxic and less desirable methanol during the polycondensation of the ethoxysilane groups.

The bipodal aminosilane can be a secondary aminosilane. Using a secondary bipodal aminosilane instead of a primary aminosilane decreases the reactivity of the polymeric stabilizer with respect to electrophilic species, in particular aldehydes. Hence, benefit agents containing high levels of aldehydes may be encapsulated with a lower propensity for adverse interactions between core-forming and shell-forming materials.

Other aminosilanes may also be used in combination with the aforementioned bipodal aminosilanes, in particular the aminosilanes described hereinabove.

The aminosilane to polymeric surfactant weight ratio can be from 0.1 to 1.1, in particular from 0.2 to 0.9, even more particularly from 0.3 to 0.7, for example 0.5.

The polymeric stabilizer can be formed by combination of a polymeric surfactant with at least one aminosilane and further a polyfunctional isocyanate. Polyfunctional isocyanates may densify the arrangement of the polymeric surfactant at the oil/water interface. Without wishing to be bound by theory, it is believed that the polyfunctional isocyanate cross-links both aminosilanes and polysaccharides by forming polyurea and polyurethane bonds.

The polyfunctional isocyanate may be selected from organic isocyanates, in which an isocyanate group is bonded to an organic residue (R—N═C═O or R—NCO). The polyfunctional isocyanate may be selected from alkyl, alicyclic, aromatic and alkylaromatic, as well as anionically modified polyfunctional isocyanates, with two or more (e.g. 3, 4, 5, etc.) isocyanate groups in a molecule.

Preferably, at least one polyfunctional isocyanate is an aromatic or an alkylaromatic polyfunctional isocyanate, the alkylaromatic polyfunctional isocyanate having preferably methylisocyanate groups attached to an aromatic ring. Both aromatic and methylisocyanate-substituted alkylaromatic polyfunctional isocyanates have a superior reactivity compared to alkyl and alicyclic polyfunctional isocyanates. Among these, 2-ethylpropane-1,2,3-triyl tris((3-(isocyanatomethyl)phenyl)carbamate) is particularly preferred, because of its tripodal nature that favors the formation of intermolecular cross-links and because of its intermediate reactivity that favors network homogeneity. This alkylaromatic polyfunctional isocyanate is commercially available under the trademark Takenate D-100 N, sold by Mitsui or under the trademark Desmodur® Quix175, sold by Covestro.

As an alternative to aromatic or alkylaromatic polyfunctional isocyanates, it may also be advantageous to add an anionically modified polyfunctional isocyanates, because of the ability of such polyfunctional isocyanates to react at the oil/water interface and even in the water phase close to the oil/water interface. A particularly suitable anionically modified polyfunctional isocyanate has Formula (III).

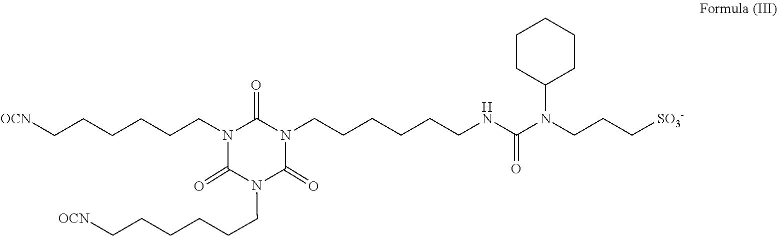

Formula (III) shows a commercially available anionically modified polyisocyanate, which is a modified isocyanurate of hexamethylene diisocyanate, sold by Covestro under the trademark Bayhydur® XP2547.

In a preferred embodiment of the present invention, the polyfunctional isocyanate is 2-ethylpropane-1,2,3-triyl tris((3-(isocyanatomethyl)phenyl)carbamate).

In a particularly preferred embodiment of the present invention, the polymeric stabilizer is formed by combination of pectin with bis(3-(triethoxysilyl)propyl)amine. Preferably, the polymeric stabilizer is formed by combination of pectin with bis(3-(triethoxysilyl)propyl)amine and 2-ethylpropane-1,2,3-triyl tris((3-(isocyanatomethyl)phenyl)carbamate). These combinations of natural polymeric surfactant and bipodal secondary aminosilane provide particularly advantageous interface stability and release properties. The stabilized interface is sufficiently impervious to effectively encapsulate the at least one benefit agent comprised in the core. The polymeric stabilizer effectively forms a shell encapsulating the benefit agent comprised in the core.

### Coacervates

As a further alternative, in one embodiment, the shell can comprise a complex coacervate formed of at least one protein and at least one polysaccharide. Such core-shell capsules have proved suitable for benefit agent encapsulation and are described, for instance in WO 1996/020612 A1, WO 2001/03825 A1 or WO 2015/150370 A1.

Cross-linking of at least one protein with a first cross-linking agent leads to the formation of a stable core composition emulsion, comprising a plurality of core composition droplets. These stabilize the emulsion in that it prevents the droplets from coalescing. These stabilized droplets act as templates on which the microencapsulation further takes place. Without wishing to be bound by theory, the cross-linking reaction of the at least one protein with the first cross-linking agent can occur as interfacial polymerization at the core composition-water phase interface, in order to form a first shell around the core composition droplets, or by formation of a simple coacervate. Also a continuum between these two processes is possible.

In particular embodiments of the present invention, the shell is formed by cross-linking of the at least one protein with the first cross-linking agent in order to form a simple coacervate.

By “coacervate” it is meant polyelectrolyte-rich droplets coexisting with an aqueous, polyelectrolyte poor continuous phase. The droplet agglomerate at interfaces to form an interfacial layer.

In the present context, the coacervate droplets agglomerate at the interface between the core composition and the aqueous phase. As a result, a stable core composition emulsion in water is formed, comprising a plurality of core composition droplets, each droplet being surrounded by coacervate droplets. These stabilize the emulsion in that it prevents the droplets from coalesce.

By “simple coacervation” is meant in the present context the formation of an interfacial layer comprising a single polyelectrolyte.

By “complex coacervation” is meant the formation of an interfacial layer comprising a mixture of polyelectrolytes.

The phenomenon of simple or complex coacervation may be observed under a light microscope, wherein it is marked by the appearance of a ring around the core composition droplet. This ring consists of the aforementioned polyelectrolyte-rich phase that has a different refractive index than the surrounding aqueous phase.

The coacervation of a polyelectrolyte is generally induced by bringing the polyelectrolyte to its isoelectric point, meaning the point where the net charge of the polyelectrolyte is zero or close to zero. This may be achieved by changing the salt concentration or the pH of the medium. In a complex coacervation, complexation occurs at the pH where one of the polyelectrolytes has an overall positive electrical charge (polycation), whereas the other polyelectrolyte has an overall negative charge (polyanion), so that the overall electrical charge of the complex is neutral.

It has been found that building first a cross-linked protein, in particular as simple coacervate, at the core composition/aqueous phase interface, followed by the complex coacervation of this cross-linked protein with a second polyelectrolyte, namely at least one polysaccharide, leads to the formation of a shell having enhanced imperviousness. In particular, the shell shows enhanced imperviousness with respect to low-molecular weight materials, i.e. materials having a molecular weight lower than 250 g/mol, such as benefit agents.

Furthermore, compared to conventional coacervate microcapsules, capsules obtained by such a process show increased stability in liquid consumer product formulations, in particular water-based consumer products, such as fabric care conditioners.

Moreover, the applicant has found that by performing the aforementioned process, it is possible to better control the size of the microcapsules, compared to conventional complex coacervation. In particular, it becomes possible to obtain microcapsules in sizes below 75 μm. This is much lower than the microcapsule sizes reported in the prior art. This is also much more advantageous as it is known that microcapsules having size below 75 μm deposit better on substrates during rinse-off applications than larger microcapsules.

The shell may be formed by cross-linking of the at least one protein and a polyfunctional nucleophile with the first cross-linking agent. It has been found that, by addition of a polyfunctional nucleophile in the cross-linking process, the stability of the capsules in the above-mentioned liquid consumer product formulations is further improved.

Proteins that are particularly suitable for this aspect of the present invention include gelatins, whey proteins, pea proteins, soy proteins, caseins and albumins, for instance bovine serum albumin.

In preferred embodiments the at least one protein is a gelatin, preferably a Type B gelatin. Type B gelatin can be obtained from the alkaline treatment of collagen and is well known for its ability to form complexes with anionic polyelectrolytes, such as negatively charged polysaccharides under mild acidic conditions.

Gelatin is usually characterized by so-called “Bloom Strength”. In the present context, the Bloom Strength refers to the rigidity of a gelatin film, as measured by so-called “Bloom Gelometer”, according to the Official Procedures of the Gelatin Manufacturers Institute of America, Inc., revised 2019, Chapter 2.1. According to this procedure, the Bloom Strength, expressed in Bloom, is equal to the weight, expressed in g, required to move vertically a standardized plunger, having a diameter of 12.5 mm, to a depth of 4 mm into a gelatin gel, which has been prepared under controlled conditions, i.e. by dissolving 6.67 wt.-% of gelatin in deionized water at 60° C., in a standardized jar, and letting the gel form for 17 hours at 10° C. The higher the weight is, the higher is the Bloom Strength of the gelatin used for making the tested gel.

In preferred embodiments the Type B gelatin has a Bloom Strength of 200 to 250 Bloom. If the Bloom Strength is too low, the gel is mechanically weak and coacervates obtained therefrom may not form a self-standing layer of gelatin-rich phase around the core composition. If the Bloom Strength is too high, then the coacervates and the gelatin-rich phase obtained therefrom may be too brittle.

The Type B gelatin can be obtainable from fish, because fish gelatin meets better acceptance within consumer than beef or pork gelatin, mainly due to health concerns, sociological context or religious rules.

Alternatively, the protein may be a vegetable protein, in particular a pea protein and/or a soy protein, which have the advantage of being vegan.

In preferred embodiments, the first cross-linking agent is a trifunctional alkylaromatic isocyanate. As mentioned before, and without wishing to be bound by theory, the applicant believes that alkylaromatic isocyanate groups have the advantage of possessing an intermediate reactivity compared to the highly reactive aromatic isocyanates and the less reactive aliphatic isocyanate.

More preferably, the trifunctional alkylaromatic isocyanate is an adduct of 2-ethylpropane-1,2,3-triol or 2-ethyl-2-(hydroxymethyl)propane-1,3-diol with 1-isocyanato-2-(isocyanatomethyl)benzene, 1-isocyanato-3-(isocyanatomethyl)benzene and/or 1-isocyanato-4-(isocyanatomethyl)-benzene.

In a particularly preferred embodiment, the trifunctional araliphatic isocyanate is an adduct of 2-ethylpropane-1,2,3-triol with 1-isocyanato-3-(isocyanatomethyl)benzene. Adducts of 2-ethylpropane-1,2,3-triol with 1-isocyanato-3-(isocyanatomethyl)benzene are available commercially under the trade names Takenate D110-N (ex Mitsui Chemicals) or Quix 175 (ex Covestro).

The polyfunctional nucleophile can be selected from the group consisting of polyamines, in particular diamines and triamines, polyols, ureas, urethanes and thiols.

In particular, the polyfunctional nucleophile can be selected from the group consisting of ethylenediamine, diethylenetriamine, triethylenetetramine, tetraethylenepentamine, pentaethylenehexamine, 1,3-diaminopropane, 1,2-diaminopropane, 1,4-diaminobutane, 1,6-diaminohexane, bis(3-aminopropyl)amine, bis(hexanethylene)triamine, tris(2-aminoethyl)amine, N,N′-bis(3-aminopropyl)-1,3-propanediamine, chitosan, nisin, arginine, lysine, ornithine, biuret, N,N,N′, N′-tetrakis(2-hydroxyethyl)ethylene diamine, N,N,N′, N′-tetrakis(2-hydroxypropyl)ethylene diamine, branched polyethylenimine, 2,4-diamino-6-hydroxypyrimidine, 2,2′-ethylenedioxy)bis (ethylamine) and 4,7,10-trioxa-1, 13-tridecanediamineresorcinol.

Furthermore, the polyfunctional nucleophile can be selected from the group consisting of guanidine, guanidine salts (for instance guanidine carbonate or guanidine hydrochloride), 1,3-diamino-guanidine, 1,1-dimethylbiguanide and 2,4,6-triaminopyrimidineguanazol.

The polyfunctional nucleophile can also be an aromatic polyamine, preferably an arylalkylamine, such as m-xylylenediamine or p-xylylenediamine.

Furthermore, the polyfunctional nucleophile can also be a cycloaliphatic diamine, such as 4,4′-diaminodicyclohexylmethane, 1,4-cyclohexanebismethylamine, isophorone diamine or 1,4-diazacycloheptane.

Moreover, the polyfunctional nucleophile can be selected from polyols, such as polyphenols and polysaccharides, in particular pentaerythritol, dipentaerythritol, glycerol, polyglycerol, ethylene glycol, polyethylene glycol, trimethylolpropane, neopentyl glycol, sorbitol, erythritol, threitol, arabitol, xylitol, ribitol, mannitol, galactitol, fucitol, iditol, inositol, volemitol, isomalt, maltitol, lactitol, maltotriitol, maltotetraitol, polyglycitol, polyphenol and tannic acid.

In preferred embodiments of the present invention, the polyfunctional nucleophile is selected from the group consisting of melamine and urea.

Preferably, the polyfunctional nucleophile is water-soluble.

In preferred embodiments of the present invention, the weight ratio of the polyfunctional nucleophile, in particular the melamine, to the at least one protein, in particular the gelatin, is from 0.01 to 1.0, preferably from 0.05 to 0.5, more preferably from 0.08 to 0.2, even more preferably from 0.1 to 0.15.

In preferred embodiments, the at least one polysaccharide preferably comprises carboxylate groups. Polysaccharides comprising carboxylate groups are particularly suitable for complex coacervation with proteins, in particular with Type B gelatin. This is due to the fact that the net electrical charge of these polysaccharides may be adjusted by adjusting the pH, so that the complexation with ampholytic proteins is facilitated. Complexation occurs at the pH where the protein has an overall positive electrical charge, whereas the polysaccharide has an overall negative charge, so that the overall electrical charge of the complex is neutral. These polysaccharides include native polysaccharides from nature and modified polysaccharides. Monovalent alkaline metal salts of these polysaccharides may also be used.

In particular, the at least one polysaccharide is selected from the group consisting of carboxymethylcellulose, gum Arabic, alginate, pectin, hyaluronic acid, xanthan gum, gellan gum, and their salts with monovalent alkaline metals. Carboxymethylcellulose, sodium carboxymethylcellulose and gum Arabic are particularly preferred.

Among the two variants of gum Arabic, i.e. gum acacia Senegal and gum acacia Seyal, gum acacia Senegal is preferred, owing to the higher level of glucuronic acid in gum acacia Senegal.

The at least one polysaccharide can be selected from the group consisting of carboxymethylcellulose and sodium carboxymethylcellulose, wherein the carboxymethylcellulose and/or the sodium carboxymethylcellulose have a molecular weight of from 50′000 to 250′000 g/mol, preferably from 75′000 to 125′000 g/mol and a degree of substitution of from 0.5 to 1.0, preferably from 0.6 to 0.8.

In preferred embodiments, the imperviousness and stability of the shell may be further improved by cross-linking of the complex coacervate with a second cross-linking agent. In particularly preferred embodiments, the second cross-linking agent is a difunctional aldehyde selected from the group consisting of succinaldehyde, glutaraldehyde, glyoxal, benzene-1,2-dialdehyde, benzene-1,3-dialdehyde, benzene-1,4-dialdehyde, piperazine-N,N-dialdehyde, and 2,2′-bipyridyl-5,5′-dialdehyde. Di-functional aldehydes are known to be effective cross-linking agents for proteins.

In this context, the weight ratio of the first cross-linking agent, in particular the trifunctional araliphatic isocyanate, to the at least one protein, in particular the gelatin, can be from 0.08 to 1.2, preferably from 0.12 to 0.8, more preferably from 0.16 to 0.6, even more preferably from 0.2 to 0.4. With such weight ratios of first cross-linking agent to protein, good stability of the microcapsules, in particular with respect to leakage, can be achieved while at the same time ensuring biodegradability.

The weight ratio of polysaccharide to protein typically depends on the nature of the polysaccharide. Without wishing to be bound by theory, it is assumed that this weight ratio depends on the degree of substitution of the polysaccharide, in particular with carboxylic or carboxylate groups, if applicable. Preferably, the weight ratio between the at least one polysaccharide and the at least one protein is from 0.05 to 0.5, preferably from 0.08 to 0.2.

Alternatively, in one embodiment, the shell comprises a hydrated polymer phase and a polymeric stabilizer at an interface between the shell and the core. In such an arrangement, the polymeric stabilizer provides an impervious encapsulating material, whereas the hydrated polymer phase provides the desired deposition and adherence to the substrate. Furthermore, without wishing to be bound by theory, it is believed that the hydrated polymer phase also provides an optimal point of attack for microbial degradation.

The polymeric stabilizer may be selected from a broad range of film-forming materials and resins. Preferably, the polymeric stabilizer is highly cross-linked, in order to decrease significantly the diffusion of the encapsulated benefit agent through the shell. Preferably the imperviousness of the shell is sufficiently high to significantly prevent the leakage of the benefit agent in extractive base, such as consumer products comprising surfactants.

In preferred embodiments of the present invention, the polymeric stabilizer is a thermosetting resin. Thermosetting resins are defined as hereinabove.

In a particularly preferred embodiment of the present invention, the polymeric stabilizer is formed by reaction of an aminosilane with a polyfunctional isocyanate. Such a polymeric stabilizer has the advantage of being highly crosslinked and susceptible of providing surface anchoring groups that can be used to immobilize additional materials to complete shell formation. These additional materials may comprise additional encapsulating materials, coatings and, as described in more details hereinafter, simple and complex coacervate, and hydrogels.

The aminosilane and polyfunctional isocyanate are defined as hereinabove.

In a preferred embodiment of the present invention, polyfunctional isocyanate is 2-ethylpropane-1,2,3-triyl tris((3-(isocyanatomethyl)phenyl)carbamate). Particularly preferably, the polymeric stabilizer is formed by reaction of bis(3-(triethoxysilyl)propyl)amine and 2-ethylpropane-1,2,3-triyl tris((3-(isocyanatomethyl)phenyl)carbamate). The combination of this particular bipodal secondary aminosilane and polyfunctional isocyanate provides advantageous interface stability and release properties. The stabilized interface is sufficiently impervious to effectively encapsulate the at least one benefit agent comprised in the core and possesses the desired surface functional groups.

In preferred embodiments of the present invention the hydrated polymer phase can be a coacervate, in particular a complex coacervate. A complex coacervate is defined as hereinabove.

In preferred embodiments of the present invention, the coacervate may be formed from a polycation and a polyanion.

Preferably, the pH is used as parameter driving the coacervation. Thus, the polycation preferably has a pH-dependent electrical charge. This is the case for polymers bearing primary, secondary and tertiary amino groups, such as polyamines, for example chitosan, and most proteins, for example gelatin. Proteins have the additional advantage of being prone to temperature-dependent structural transitions that may also be used to control the morphology of the coacervates. In particular, varying the temperature of some proteins may induce the formation of secondary, tertiary or quaternary structures of the protein that may also be used to control the properties of the coacervate.

Chitosan has the advantage of being derived from chitin, which is a natural polymer.

In preferred embodiments of the present invention, the polycation is selected from the group consisting of proteins and chitosan.

More particularly, the polycation can be a protein selected from the group consisting of gelatin, casein, albumin, polylysine, soy proteins, pea proteins, rice proteins and hemp proteins.

In particularly preferred embodiments of the present invention, the at least one protein is a gelatin, even more preferably a Type B gelatin. Type B gelatin is defined as hereinabove.

The polycation may be a denaturated protein. In the contrary to native proteins, denaturated proteins have been deprived from their ability to form secondary, tertiary or quaternary structures and are essentially amorphous. Such amorphous proteins may form more impervious films compared to native proteins and therefore also contribute to the encapsulating power of the shell. Denaturation may be achieved by treating the protein with chemical or physical means, such as acid or alkaline treatment, heat or exposure to hydrogen bond disrupting agents.

In cases where the polycation is chitosan, the chitosan can have a molecular weight between 3′000 and 1′000′000 g/mol, more particularly between 10′000 and 500′000 g/mol, still more particularly between 30′000 and 300′000 g/mol.

The polyanion may be any negatively charged polymer. However, as the pH is preferably used to control coacervation, it may be more advantageous that the electrical charge of the polymer is pH-dependent. Such polymer may be selected from polymers having pendent carboxylic groups, such as methacrylic acid and acrylic acid polymers and copolymers, hydrolyzed maleic anhydride copolymers and polysaccharides bearing carboxylic groups.

In preferred embodiments of the present invention, the polyanion is a polysaccharide comprising carboxylate groups and/or sulfate groups. Polysaccharide comprising carboxylate groups are as defined hereinabove.

The hydrated polymer phase can be a hydrogel.

In context of the present invention, a “hydrogel” is a three-dimensional (3D) network of hydrophilic polymers that can swell in water, while maintaining the structure due to chemical or physical cross-linking of individual polymer chains.

Such a hydrogel can be formed by several methods at interfaces, especially by self-assembly of polyelectrolytes around existing interfaces, covalent grafting of pre-formed hydrogel particles in solution, polymerization of hydrosoluble monomers initiated at the interface and phase separation of water soluble macromolecules onto the interface.

To avoid any ambiguity, in context of the present invention, a coacervate, especially a complex coacervate, which is cross-liked, in particular by covalent bonds, is considered as a hydrogel.

The applicant has found that the use of hydrogels particularly enhances both the deposition and adherence of microcapsules on substrates, in particular on fabrics.

The hydrogel can be interlinked with the polymeric stabilizer, in particular via the functional groups present on the surface of this stabilizer.

This allows the locking of the hydrogel layer onto the polymeric stabilizer present at droplet interface, making the shell composed of a polymer composite, instead of only a blend.

Both hydrogel cross-linking and hydrogel interlinking with the polymeric stabilizer may be performed sequentially or simultaneously.

In preferred embodiments of the present invention, the hydrogel is a crosslinked coacervate, in particular a complex coacervate crosslinked with polyfunctional aldehyde, more particularly a difunctional aldehyde selected from the group consisting of succinaldehyde, glutaraldehyde, glyoxal, benzene-1,2-dialdehyde, benzene-1,3-dialdehyde, benzene-1,4-dialdehyde, piperazine-N,N-dialdehyde and 2,2′-bipyridyl-5,5′-dialdehyde. Difunctional aldehydes are known to be effective cross-linking agents for proteins.

The hydrogel can be thermosensitive and possess a gelation temperature, in particular between 20° C. and 50° C., preferably between 25° C. and 40° C. When using such a hydrogel, the deposition performance of the capsules on fabic can increase, when washing the fabric at a temperature which is above hydrogel gelation temperature.

The shell can be further stabilized with a stabilizing agent. The stabilizing agent is defined as hereinabove.

In context of the present invention, the shell of the microcapsules can be made of a biodegradable material or a non-biodegradable material. In one embodiment, the microcapsules are made of a biodegradable material.

In preferred embodiments of the present invention, the volume median diameter Dv(50) of the plurality of core-shell microcapsules is from 1 to 100 μm, preferably 5 to 75 μm, more preferably 8 to 60 μm, even more preferably 10 to 30 μm. Microcapsules having volume median diameter in the range from 10 to 30 μm show optimal deposition on various substrates, such as fabrics and hair.

The resultant encapsulated composition, presented in the form of a slurry of microcapsules suspended in an aqueous suspending medium, may be incorporated as such in a consumer product base. If desired, however, the slurry may be dried to present the encapsulated composition in dry powder form. Drying of a slurry of microcapsules is conventional, and may be carried out according techniques known in the art, such as spray-drying, evaporation, lyophilization or use of a desiccant. Typically, as is conventional in the art, dried microcapsules will be dispersed or suspended in a suitable powder, such as powdered silica, which can act as a bulking agent or flow aid. Such suitable powder may be added to the encapsulated composition before, during or after the drying step.

In a microcapsule composition according to the present invention, the proportion of the benefit agent can be between about 10 to about 50 wt.-%, preferably between about 20 to about 47.5 wt.-%, even more preferably between about 30 to about 45 wt.-%, relative to the total weight of the microcapsule composition.

The proportion of the microcapsule composition as described herein above may be between about 1 wt.-% to about 30 wt.-%, preferably between about 1.5 wt.-% to about 20 wt.-%, more preferably between about 2 wt.-% to about 10 wt.-%, relative to the total weight of the solid composition.

### Crystallization Additive

In the present context, the role of the crystallization agent is to speed up the crystallization process.

Suitable crystallization additive are selected from the group consisting of polyols, di- or polysaccharides, starch derivatives and organic acids.

Examples of suitable polyols are sorbitol and maltitol. Examples of suitable di-or polysaccharides are sucralose, saccharose and fructose. Examples of suitable organic acids are citric acid, tartaric acid and formic acid.

Optionally, the crystallization additive is citric acid. Citric acid is a cost effective bio-based and biodegradable compound capable to influence the kinetics of crystallization of the solid carrier and thus the solidification of the composition.

If a crystallization additive is employed, its proportion in the solid composition may be between about 0.1 wt.-% to about 5.0 wt.-%, preferably between about 0.2 wt.-% to about 4.0 wt.-%, even more preferably between about 0.5 wt.-% to about 3.0 wt.-%, relative to the total weight of the solid composition.

### Dye

Optionally, the solid composition may comprise a dye, especially selected from the group consisting of Carotenoids (E160), Xanthins (E161), Saffron (E164), Chlorophylls (E140), copper complexes of Chlorophylls and/or Chlorophyllins (E141), Anthocyanins (E163), Carmine (E120), Curcumin (E100) and their derivatives, or a visual modifier, especially inorganic and organic pigments like mica powders or titanium dioxide.

If a dye or visual modifier is used, the proportion of the same can be between about 0.01 wt.-% to about 5.0 wt.-%, preferably about 0.1 wt.-% to about 4.0 wt.-%, more preferably between about 0.2 wt.-% to about 2.0 wt.-%, relative to the total weight of the solid composition.

### Filler

Optionally, the solid composition may comprise a filler, in particular a filler selected from the group consisting of silica, sodium carbonate, sodium bicarbonate, magnesium aluminum silicate, bentonite, ion exchange resin, sodium dodecyl sulphate, and combinations thereof.

If a filler is used, its proportion in the solid composition may be between about 0.1 wt.-% to about 5 wt.-%, preferably between about 1 wt.-% to 4 wt.-%, relative to the total weight of the solid scent booster composition.

### Non-Encapsulated Fragrance

Optionally, the solid composition may comprise a non-encapsulated fragrance ingredient.

The fragrance ingredient is as defined hereinabove.

The non-encapsulated fragrance ingredient can be identical or different from the fragrance ingredient as a benefit agent used in the microcapsule composition as described herein above. This results in a modulated release of the same or of different odor impressions, depending on whether the encapsulate is exposed to moisture or mechanical stresses. In particular, a sequential release of the fragrance may be envisioned.

The solid compositions of the present invention allow for fragrance ingredient release either through activation by mechanical action or by moisture, for instance in deodorant or antiperspirant applications. But such compositions are also particularly useful when employed as fragrance delivery means in consumer products that require, for delivering optimal perfumery benefits, core-shell microcapsules to adhere to a substrate on which they are applied, for instance laundry detergents.

The non-encapsulated fragrance ingredient can comprise, preferably consists of, at least one, preferably at least two, more preferably at least four, even more preferably at least eight, even still more preferably at least sixteen, biodegradable ingredient(s). The biodegradable ingredient(s) can be present at a total concentration of at least 75 wt.-%, preferably at least 80 wt.-%, more preferably at least 85 wt.-%, even more preferably at least 90 wt.-%, even still more preferably at least 95 wt.-%, relative to the total weight of the fragrance ingredient. The biodegradable ingredient(s) can be selected from the groups as defined hereinabove.

If a non-encapsulated fragrance ingredient is used, its proportion can be between about 0.1 wt.-% to about 29 wt.-%, preferably between about 1 wt.-% to about 15 wt.-%, more preferably between about 10 wt.-% to about 15 wt.-%, relative to the total weight of the solid composition.

In one embodiment, at least one, preferably at least two, preferably at least three, preferably at least four, preferably all components b), c), d), e) and f) of the composition as defined herein are biodegradable.

In one embodiment, the composition is vegan.

In one embodiment, the composition is sustainable,

In one embodiment, the composition is bio-based.

In one embodiment, the composition is Halal or Kosher.

The solid composition of the present invention can be in the form of a plurality of pastilles. Each of the pastilles can have a mass of 0.01 g to 15.0 g, preferably 0.01 g to 5.0 g, more preferably 0.015 g to 2.0 g. Furthermore, each of the pastilles can have a maximum dimension of less than 50 mm, preferably less than 20 mm, more preferably less than 8 mm. Moreover, each of the pastilles can have a shape selected from the group consisting of polyhedron, polygonal prism, spherical, hemispherical, compressed hemispherical, lentil shaped and oblong.

### Method

In a second aspect, a method of making a composition as described herein is provided.

The method comprises the following steps:
i) heating a solid, water-soluble, biodegradable carrier to a temperature of about the meting point of the carrier, to obtain a melted composition of carrier;
ii) optionally, adding a crystallization additive to the melted composition of carrier obtained in step i);
iii) adding a microcapsule composition, comprising a polymer encapsulating a benefit agent, wherein the benefit agent is encapsulated exclusively in core-shell microcapsules comprising a core and a shell surrounding the core to the melted compositions obtained in step i) or in step ii);
iv) optionally, adding to the composition of step iii) a dye or visual modifier;
v) optionally, adding to the composition of step iv) a filler;
vi) optionally, adding to the composition of step v) at least one non-encapsulated fragrance ingredient;
vii) cooling the composition obtained in step iii), step iv), step v) or step vi) to room temperature.

In one embodiment, a non-encapsulated fragrance ingredient is added to the microcapsule composition comprising a polymer encapsulating a benefit agent of step iii) prior to adding the microcapsule composition to the melted composition obtained in step i) or step ii).

The advantage of employing a carrier with a lower melting point is that the energy required for the production of the composition is minimal.

### Consumer Product

The present invention also relates to a consumer product comprising a solid composition as described hereinabove. The consumer product may be selected from the group consisting of personal care, fabric care, household (home) care and pet care products.

Suitable home care products include air care compositions, hard surface cleaners, heavy duty detergents and detergent powders, carpet cleaners.

Suitable personal care products include deodorant compositions, bath salts, cleansing compositions (such as soap bars), oral care compositions, antiperspirant compositions, skin care products.

Suitable fabric care compositions include laundry care detergents, fabric refreshers, scent boosters.

In one embodiment, the consumer product is a solid scent booster. Scent booster compositions according to the present invention are particularly attractive, especially with regard to consumer appeal, due to their reduced environmental impact. The compositions as described herein above have proven very suitable for the manufacture of solid scent boosters, as they allow for the provision of perfume compositions in dry form and with very low water content.

The present invention is further illustrated by means of the following non-limiting examples:

### EXAMPLE 1: PREPARATION OF XYLITOL-BASED SOLID SCENT BOOSTER COMPOSITION WITH CORE-SHELL ENCAPSULATED FRAGRANCE (NO NON-ENCAPSULATED FRAGRANCE INGREDIENT)

A solid scent booster composition was prepared by performing the following steps:
a) Xylitol powder (100 g) is added inside a 150 ml beaker and is stirred for 5 min at room temperature;
b) The temperature is increased to 95° C. and maintained until the mixture is completely molten;
c) 3.75 g of 55% liquid citric acid solution is added to the hot melt solution of step b):
d) The hot melt is stirred at 50 RPM for 5 min;
e) 2 g of a slurry of core-shell microcapsules (prepared according to WO 2017/001672) is added and stirring is maintained until capsules are homogeneously dispersed;
f) The mixture is pumped and distributed as hemispheric droplets on a silicone substrate;
g) The hemispheric droplets are allowed to crystallize at room temperature for 4 hours;
h) Solid scent booster pastilles containing encapsulated fragrance are obtained.

### EXAMPLE 2: PREPARATION OF XYLITOL-BASED SOLID SCENT BOOSTER COMPOSITION WITH BOTH CORE-SHELL ENCAPSULATED FRAGRANCE AND NON-ENCAPSULATED FRAGRANCE INGREDIENT

A solid scent booster composition was prepared by performing the following steps:
a) Xylitol powder (100 g) is added inside a 150 ml beaker and is stirred for 5 min at room temperature;
b) The temperature is increased to 95° C. and maintained until the mixture is completely molten;
c) 3.75 g of 55% liquid citric acid solution is added to the hot melt solution of step b):
d) The hot melt is stirred at 50 RPM for 5 min;
e) A mixture of 1.4 g of a slurry of core-shell microcapsules (prepared according to WO 2017/001672) and 4 g of free non-encapsulated fragrance is added and stirring is maintained until capsules are homogeneously dispersed;
f) The mixture is pumped and distributed as hemispheric droplets on a silicone substrate;

g) The hemispheric droplets are allowed to crystallize at room temperature for 4 hours;
h) Solid scent booster pastilles containing encapsulated fragrance are obtained.

### EXAMPLE 3 (COMPARATIVE): PREPARATION OF XYLITOL-BASED SOLID SCENT BOOSTER COMPOSITION WITH NON-ENCAPSULATED FRAGRANCE INGREDIENTS BUT NO CORE-SHELL ENCAPSULATED FRAGRANCE

A solid scent booster composition was prepared by performing the following steps:
a) Xylitol powder (100 g) is added inside a 150 ml beaker and is stirred for 5 min at room temperature;
b) The temperature is increased to 95° C. and maintained until the mixture is completely molten;
c) 3.75 g of 55% liquid citric acid solution is added to the hot melt solution of step b):

d) The hot melt is stirred at 50 RPM for 5 min;
e) 5 g of free non-encapsulated fragrance is added and stirring is maintained;
f) The mixture is pumped and distributed as hemispheric droplets on a silicone substrate;
g) The hemispheric droplets are allowed to crystallize at room temperature for 4 hours;
h) Solid scent booster pastilles are obtained.

### EXAMPLE 4 (COMPARATIVE): PREPARATION OF PEG-BASED SOLID SCENT BOOSTER COMPOSITION WITH CORE-SHELL ENCAPSULATED FRAGRANCE

a) PEG 6000 powder (100 g) is added inside a 150 ml beaker and is stirred for 5 min at room temperature;
b) The temperature is increased to 95° C. and maintained until the mixture is completely molten;
c) 2 g of Core of core shell slurry is added and stirring is maintained until capsules are homogeneously dispersed
d) The mixture is pumped and distributed as hemispheric droplets on a silicone substrate;
e) The hemispheric droplets are allowed to crystallize at room temperature for 4 hours;
f) Solid scent booster pastilles containing encapsulated fragrance are obtained.

### EXAMPLE 5: COMPARISON OF OLFACTIVE PERFORMANCE

The olfactive performance of the scent booster was assessed by a panel of 4 experts who rated the odor intensity on a scale of 1-5 (1=barely noticeable, 2=weak, 3=medium, 4=strong and 5=very strong). When relevant, qualitative comments on the perceived odor direction were recorded.

The scent boosters of Examples 1 to 4 were tested in the following conditions:

18 g of solid scent booster was placed inside the detergent dispenser of a washing machine containing 2 kg of wash fabric load. The washing temperature was 40° C. The “wet” (i.e. out-of-the-washing machine) odor intensity was assessed on wet fabrics within 5 min after having removed the fabrics from the machine. The pre-rub olfactive evaluation was performed after line drying the fabrics for 24 h at room temperature. The post-rub evaluation was performed by gently rubbing one part of the fabrics. The performance of the scent boosters is shown in Table 1.

TABLE 1
Olfactive performance of the compositions of Examples 1 to 4

| | Intensity | Pre-rub | Post-rub |
|---|---|---|---|
| Example | on wet | intensity on dry | intensity on dry |
| 1 | 1.5 | 3 | >2 |
| 2 | 3.95 | 3 | 2.75 |
| 3 | 3.75 | 0.5 | 0.25 |
| 4 | 1 | 2 | >1.5 |

From the table it can be observed that the xylitol-based compositions comprising encapsulated fragrance encapsulated in core-shell microcapsules (Examples 1 and 2) showed significantly higher pre-and post-rub intensities than the xylitol-based composition lacking encapsulated fragrance (Example 3), showing also superior performance compared to a PEG-based scent booster employing encapsulated fragrance (Example 4).

1. A solid composition comprising:
a) a solid, water-soluble, biodegradable carrier;
b) a microcapsule composition comprising a polymer encapsulating a benefit agent, wherein the benefit agent is encapsulated in core-shell microcapsules comprising a core and a shell surrounding the core;
c) optionally, a crystallization additive;
d) optionally, a dye;
e) optionally, a filler; and
f) optionally, at least one non-encapsulated fragrance ingredient,
wherein, the composition excludes water soluble polymrs selected form: starch, modified starch, maltodxtrins, polysaccharides, carbohydrates, chitosan, gum arabic, polyethylen glycol (PEG), polyvinly pyrrolidone (PVP), polyvinyl alcohol, acrylamides, acrylates, polyacrylic acid and related, maleic anhydride copolymers, amine-functional polymers, vinyl ethers, styrenes, polystryrenessulfonates, vinyl acids, ehtylene glycol-propylene glycol block copolymers, and mixutres thereof.

2. The composition according to claim 1, wherein the carrier is a sugar alcohol or a mixture thereof.

3. The composition according to claim 1, wherein the melting point of the carrier is from about 70° C. to about 200° C.

4. The composition according to claim 1, wherein the shell of the microcapsule comprises a melamine-formaldehyde polymer, an urea-formaldehyde polymer, a polyurea or polyurethane polymer, a polyamide, a polyacrylate, a polycarbonate, a polymeric stabilizer that is formed by combination of a polymeric surfactant with at least one aminosilane, a complex coacervate formed by cross-linking of at least one protein with a first cross-linking agent and at least one polysaccharide, or a hydrated polymer and a polymeric stabilizer formed by reaction of an aminosilane with a polyfunctional isocyanate.

5. The composition according to claim 1, wherein the benefit agent is selected from the group consisting of fragrance ingredients, cosmetic ingredients, bioactive agents, substrate enhancers, enzymes, dyes, pigments and nutraceuticals.

6. The composition according to claim 1, wherein the crystallization additive is selected from the group consisting of a polyol, a starch derivative and an organic acid.

7. The composition according to claim 1, wherein the dye is selected from the group consisting of Carotenoids (E160), Xanthins (E161), Saffron (E164), Chlorophylls (E140), copper complexes of Chlorophylls and/or Chlorophyllins (E141), Anthocyanins (E163), Carmine (E120), Curcumin (E100) and their derivatives.

8. The composition according to claim 1, wherein the filler is selected from the group consisting of silica, sodium carbonate, sodium bicarbonate, magnesium aluminum silicate and bentonite.

9. The composition according to claim 1, wherein components b) is biodegradable.

10. The composition according to claim 1, comprising
a) from about 70% to about 99% w/w of a solid, water-soluble, biodegradable carrier;
b) from about 1% to about 30% w/w of a microcapsule composition, comprising a polymer material encapsulating a benefit agent, wherein the benefit agent is exclusively encapsulated in core-shell microcapsules comprising a core and a shell surrounding the core;
c) from 0 to about 5% w/w of a crystallization additive;
d) from 0 to about 5% w/w of a dye or visual modifier;
e) from 0 to about 5% w/w of a filler; and
f) from 0 to about 29% w/w of at least one non-encapsulated fragrance ingredient.

11. A method of making a composition according to claim 1, comprising the steps of
i) heating a solid, water-soluble, biodegradable carrier to a temperature of about the meting point of the carrier, to obtain a melted composition of carrier;
iii) adding a microcapsule composition, comprising a polymer encapsulating a benefit agent, wherein the benefit agent is encapsulated in core-shell microcapsules comprising a core and a shell surrounding the core to the melted compositions obtained in step i)
vii) cooling the composition obtained in step iii) to room temperature.

12. A method of making a composition according to claim 11, wherein the microcapsule composition in step iii) further comprises a non-encapsulated fragrance ingredient.

13. A consumer product comprising the composition according to claim 1, wherein the consumer product is selected from the group consisting of a personal care product, a fabric care product, a home care product or a pet care product.

14. A consumer product according to claim 13, wherein the consumer product is a scent booster.

15. A method of enhancing the performance of the benefit agent in a consumer product, the method comprising the step of including the composition of claim 1 in the consumer product.

16. The composition according to claim 5, wherein the benefit agent is a fragrance ingredient.

17. A method of making a composition according to claim 10, comprising the steps of:
i) heating a solid, water-soluble, biodegradable carrier to a temperature of about the meting point of the carrier, to obtain a melted composition of carrier;
ii) adding a crystallization additive to the melted composition of carrier obtained in step i);
iii) adding a microcapsule composition, comprising a polymer encapsulating a benefit agent, wherein the benefit agent is encapsulated in core-shell microcapsules comprising a core and a shell surrounding the core to the melted compositions obtained in step i) or in step ii);
iv) adding to the composition of step iii) a dye or visual modifier;
v) adding to the composition of step iii) or step iv) a filler;
vi) adding to the composition of step iii), step iv) or step v) at least one non-encapsulated fragrance ingredient;
vii) cooling the composition obtained in step iii), step iv), step v) or step vi) to room temperature.

18. The solid composition of claim 1, consisting of:
a) a solid, water-soluble, biodegradable carrier;
b) a microcapsule composition comprising a polymer encapsulating a benefit agent, wherein the benefit agent is encapsulated in core-shell microcapsules comprising a core and a shell surrounding the core;
c) optionally, a crystallization additive;
d) optionally, a dye;
e) optionally, a filler; and
f) optionally, at least one non-encapsulated fragrance.

19. The composition according to claim 2, wherein the carrier is a sugar alcohol or selected from xylitol, sorbitol, arabitol, mannitol, erythritol and mixtures thereof.

20. A method of enhancing the performance of the benefit agent in a consumer product, the method comprising the step of including the composition formed by the method of claim 11 in the consumer product.
